SEQUENCE LISTING (8 sequences)

SEQ ID NO: 1 - protein, 407 aa, Homo sapien
MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 2 - protein, 350 aa, C. elegans
MFMASSLPVFIFLLSLPHGLTFNCTNSGVCIEKMKQHRTEYLKNEILDQLNMKEAPKGLKPMDPEMKSVYLEMYRDLLEKDEQDMGVEMSFYTAKDPSYGENPSQLVAKFDVTNDLERSDILQATLTVSIEIPAKDSGMLQDVQVQVYEKNEDGSMGEMVTSGIFATKGSERISIQLPIDTVKSWFTISPIQGIFVKAMLDGRNVALHPQQTTADVDNMRLQLSTRPKGSRKRRSHAKPVCNAEAQSKGCCLYDLEIEFEKIGWDWIVAPPRYNAYMCRGDCHYNAHHFNLAETGHSKIMRAAHKVSNPEIGYCCHPTEYDYIKLIYVNRDGRVSIANVNGMIAKKCGCS

SEQ ID NO: 3 - protein, 121 aa, A. gambiae
IEIEFRDAPKKRIKRNLSLNCDENANETRCCRYPLTVDFEKFGWDWIIAPKRYEAWYCAGECMISFLPKYEHTHVMQLSTSAIPCCSPKKMNSIRLLYFDMSYNVIYSTIPNMVVEKCSCS

SEQ ID NO: 4 - protein, 390 aa, D. rerio
MKRYNFLLCLTVLISLGLSGSDEPNLFLAPLSEMSSDIGVSLFDVDDVESSECSACVWREQSKVLRLETIKSQILSKLRLKQAPNISREVVNQLLPKAPPLQQLLDHHDFQGDASSLEDFIDADEYHATTESVITMASEPEPLVQVDGKPTCCFFKFSPKLMFTKVLKAQLWVYLQPLKQTSTVYLQILRLKPITEQGSRHIRIRSLKIELDSQAGHWQSIDFKHVLQNWFKQPHTNWGIDINAYDESGNDLAVTSLGPGEEGLQPFLEVKILETTKRSRRNLGLDCDEHSTESRCCRYPLTVDFEAFGWDWIIAPKRYKANYCSGQCEYMFMQKYPHTHLVQHANPRGSAGPCCTPTKMSPINMLYFNDKQQIIHGKIPGMVVDRCGCS

SEQ ID NO: 5 - protein, 375 aa, B. taurus
AAAAAAAAAGAGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIDLHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 6 - protein, 405 aa, M. musculus
MVLAAPLLLGFLLLALELRPRGEAAEGPAAAAAAAAAAAGVGGERSSRPAPSAPPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 7 - protein, 407 aa, P. troglodytes; identical to SEQ ID NO: 1

SEQ ID NO: 8 - protein, 405 aa, R. norvegicus
MVLAAPLLLGFLLLALELRPRGEAAEGPAAAAAAAAAAAGVGGERSSRPAPSAAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

(19) United States
(12) Patent Application Publication
(10) Pub. No.: US 2016/0120945 A1
(43) Pub. Date: May 5, 2016

(54) Thymic Regeneration

(71) Applicants: Amy WAGERS, (US); JOSLIN DIABETES CENTER, Boston, MA (US); THE BRIGHAM AND WOMEN'S HOSPITAL, INC., Boston, MA (US)

(72) Inventors: Thomas Serwold, Cambridge, MA (US); Richard T. Lee, Weston, MA (US); Amy Wagers, Brookline, MA (US)

(73) Assignees: THE BRIGHAM AND WOMEN'S HOSPITAL, INC., Boston, MA (US); JOSLIN DIABETES CENTER, Boston, MA (US)

(21) Appl. No.: 14/768,181

(22) PCT Filed: Feb. 14, 2014
(86) PCT No.: PCT/US14/16399

§ 371 Date: Aug. 14, 2015

Related U.S. Application Data
(60) Provisional application No. 61/765,535, filed on Feb. 15, 2013.

(51) Int. Cl. A61K 38/18; A61K 45/06

(52) U.S. Cl. CPC A61K 38/1875; A61K 45/06

(57) ABSTRACT

Methods for regenerating thymic tissues, e.g., for the treatment of subjects who have thymic insufficiency, i.e., whose thymic tissues are absent or atrophied due to illness, age, or injury, by administration of growth differentiation factor 11 (GDF11).

### CLAIM OF PRIORITY

The present application claims the benefit of U.S. Provisional Patent Application No. 61/765,535, filed Feb. 15, 2013. The entire contents of the foregoing are incorporated by reference herein.

### TECHNICAL FIELD

This invention relates to methods for regenerating thymic tissues, e.g., for the treatment of subjects who have thymic insufficiency, i.e., whose thymic tissues are absent or atrophied due to illness, age, or injury, by administration of growth differentiation factor 11 (GDF11).

### BACKGROUND

During the course of a normal lifetime, the thymus undergoes significant changes in size (Haynes, B. F., G. D. Sempowski, A. F. Wells, and L. P. Hale, 2000. The human thymus during aging. Immunol Res 22:253-2611). It reaches a maximum size before puberty, when it is roughly as big as the heart, and then shrinks steadily with age. By the time an individual reaches their 6th or 7th decade of life, the thymus is so small that it is difficult to discern from surrounding adipose tissue, and its ability to produce new T cells is diminished dramatically, or even completely halted. This diminution of thymus size and thymic T cell production, which can also be adversely affected by other health issues, leads to an immunocompromised state which precludes effective immune responses against pathogens.

### SUMMARY

At least in part, the present invention is based on the discovery that administration of GDF11 can induce the growth of thymic tissues, e.g., by inducing the proliferation of thymic epithelial cells, leading to a gain or recovery of thymic function.

Thus, in one aspect the invention provides methods for treating thymic insufficiency in a mammalian subject. The methods include administering to the subject a therapeutically effective amount of a composition comprising a growth differentiation factor 11 (GDF11) polypeptide.

In another aspect, the invention features methods for treating thymic insufficiency in a mammalian subject. The methods include administering to the subject a therapeutically effective amount of a composition comprising a nucleic acid encoding a growth differentiation factor 11 (GDF11) polypeptide.

In yet another aspect, the invention provides methods for treating thymic insufficiency in a mammalian subject. The methods include administering to the subject a therapeutically effective amount of a composition comprising a cell expressing an exogenous nucleic acid encoding a growth differentiation factor 11 (GDF11) polypeptide.

Also provided herein is the use of a GDF11 polypeptide or nucleic acid encoding a GDF11 polypeptide for treating thymic insufficiency in a subject, or for the manufacture of a medicament for the treatment of thymic insufficiency in a subject.

In some embodiments of the methods or uses the polypeptide comprises an amino acid sequence that is at least 80%, 90%, or 95% identical to the full length of SEQ ID NO:1, or at least 80%, 90%, or 95% identical to amino acids 25-407 or 299-407 of SEQ ID NO:1.

In some embodiments of the methods or uses described herein, the subject is a human. In some embodiments of the methods or uses described herein, the subject has lymphopenia (reduced numbers of T cells), e.g., levels of CD4+ T cells that are persistently (e.g., over a period of weeks to months) below a threshold (e.g., normal) level, e.g., below about 50, 100, 200, 300, 400, or 500 cells per mm³ of whole blood.

In some embodiments of the methods or uses described herein, the subject has cancer. In some embodiments of the methods or uses described herein, the subject has been treated with a thymotoxic chemotherapy.

In some embodiments, the methods described herein include administering an immunotherapy, e.g., a cancer immunotherapy, to the subject. In some embodiments, the immunotherapy is selected from the group consisting of administration of dendritic cells or peptides with adjuvant; DNA-based vaccines; cytokines (eg., IL-2); cyclophosphamide; anti-interleukin-2R immunotoxins; and antibodies, virus-based vaccines (e.g., adenovirus), formulations of Toll-like Receptor or RIG-I-like receptor ligands, Adoptive T cell therapy or other cell types.

In some embodiments of the methods or uses described herein, the subject has a chronic illness, e.g., a chronic viral illness such as infection with Human Immunodeficiency Virus (HIV), acquired immune deficiency syndrome (AIDS), and AIDS-related complexes; chronic hepatitis (e.g., infection with Hepatitis C or Hepatitis B virus); subacute sclerosing panencephalitis (chronic measles encephalitis); chronic papovavirus encephalitis (progressive multifocat leukoencephalopathy); and Epstein-Barr virus infection.

In some embodiments of the methods or uses described herein, the subject has an autoimmune disease associated with reduced numbers or reduced repertoire of T cells.

In some embodiments of the methods or uses described herein, the subject has experienced trauma to the thymic region or has had a surgical procedure that decreased the size of the thymus, e.g., cardiothoracic surgery (e.g., in neonates).

In some embodiments of the methods or uses described herein, the subject is over the age of 50, e.g., is at least 60, 70, 80, or 90 years of age.

In some embodiments of the methods or uses described herein, the GDF11 is administered in combination with a treatment comprising castration; administration of keratinocyte growth factor; administration of ghrelin; administration of human growth hormone; and administration of interleukin-22.

Unless otherwise defined, technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a graph showing that GDF11 increases thymic cellularity in aged mice. Aged female mice (24 months old) were either given regular injections of GDF11. for one month, or left untreated. At the end of the treatment period, thymuses were removed and total cell numbers of each thymus were determined. Each point represents the total thymus cellularity of one aged mouse, either treated with GDF11 (squares) or left untreated (circles).

FIGS. 2A-C are images showing that thymic architecture of GDF11-induced thymuses is normal. Each panel shows a thymus from a distinct GDF11 treated, aged, mouse. Each thymus contains a well-defined cortex (dark grey, C), and a medulla region (lighter/medium grey, M).

FIG. 3 is a multiple alignment of GDF11 sequences from several species. See Table 1 for the list of species.

FIG. 4A is a line graph showing expression of GDF11 receptors in thymic epithelial cells.

FIG. 4B is a chart showing expression of GDF11 receptors in hematopoietic cells within the thymus.

### DETAILED DESCRIPTION

During the course of a normal lifetime, the thymus undergoes significant changes in size (Haynes et al., 2000). In mice and humans, the thymus is maximal in size at a young age, and begins to decline in size at around the time of puberty (Tosi et al., Clin Exp Immunol 47,497-504 (1982); Hale et al., Proc Natl Acad Sci USA 103, 8447-8452 (2006)). In humans beyond the 6th or 7th decade of life, the remaining thymic tissue is profoundly diminished, resulting in a loss of T cell production and contributing to an immunocompromised state (Haynes et al., 2000).

The cellular and molecular mechanisms that drive normal, age-related thymus atrophy have not been previously defined, and while certain regimens and cytokines have been identified that can stimulate thymus regeneration to some extent, there remains a need for understanding the mechanisms that control thymus involution and regeneration (Heng et al., Curr Opin Pharmacol 10, 425-433 (2010)).

The most dramatic enhancement of thymus regeneration is seen in aged male mice that have undergone castration. The thymuses of old (male) mice grow back to nearly the same size as young thymuses (Sutherland et al., 2005. J Immunol 175: 2741-2753). This finding indicates that the thymus is capable of extensive regeneration. In humans, reversible sex steroid ablation may be a feasible approach for inducing thymus regeneration, and initial results show some promise (Sutherland et al., 2005. J Immunol 175:2741-2753). However, this approach may be less beneficial in females, and has associated risks.

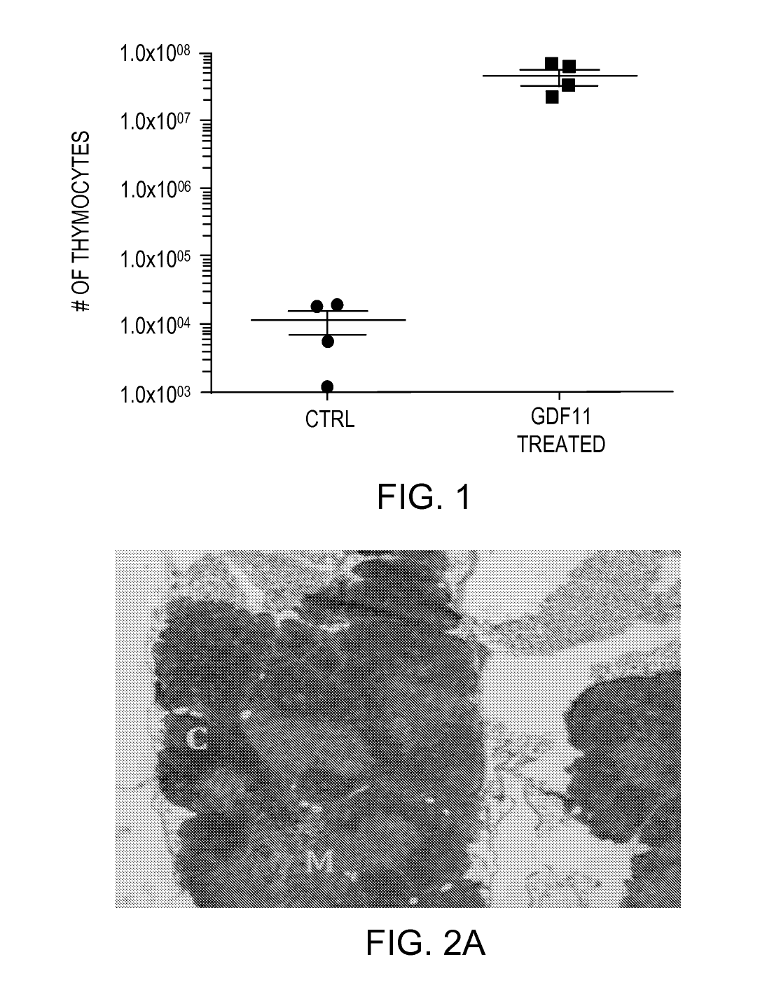

As described herein, GDF11, a TGFbeta family member, is a potent inducer of thymus regeneration in aged mice. 24-month old female mice (roughly equivalent to 72-years of age in humans) that received daily injections of GDF11 for one month showed a dramatic increase in thymic cellularity compared to age-matched, control-injected mice (FIG. 1). The thymuses of GDF11 treated mice regenerated medullary and cortical thymic zones, and had a normal representation of thymocyte subpopulations as determined by histology and flow cytometry. Therefore, GDF11 drives thymic regeneration in aged mammals.

Methods of Treatment

The methods described herein include methods for the treatment of subjects having conditions and disorders associated with thymic insufficiency. In some embodiments, the subject has reduced numbers of T cells, e.g., levels of CD4+ T and/or levels of naive (CD45RA+CD62L+) T cells, that are persistently (e.g., over a period of weeks to months) below a threshold (e.g., normal) level, e.g., below about 50, 100, 200, 300, 400, or 500 cells/mm³ of whole blood, e.g., less than 50 naive T cells/mm³, or their naive T cells comprise less than 5% of total cells by flow cytometry. Alternatively or in addition, thymus deficiency can be diagnosed based on a low number of recent thymic emigrating T cells via PCR-based measurement of TCR-excision circles (e.g., as described in Geenen et al., (2003). J. Endocrinol. 176, 305-311).

In some embodiments, the subject has been exposed to a toxin that affect thymic size or function, e.g., organotin compounds, glucocorticosteroids, 2,3,7,8-tetrachlorodibenzo-p-dioxin, or cyclosporine (see, e.g., Schuurman et al., Int J Immunopharmacol. April 1992; 14(3):369-75). In some embodiments, the subject has cancer, and has been treated with a chemotherapeutic agent that is thymotoxic. Toxicity or lesion in thymus has been reported in the following cancer treatments: Pre-bone marrow transplantation conditioning, chemotherapy, radiotherapy (Heng et al., Curr Opin Pharmacol 10(4):425-33, 2010); cisplatin (Rebillard et al., Oncogene. 27(51):6590-5, 2008); cyclophosphamide (CPA) (Zusman et al., In Vivo. 16(6):567-76, 2002); NAVELBINE® i.v. (Vinorelbine) (Su et al., Int J Pharm 411(1-2):188-96, 2011); nucleoside-based analogues (Belinsky et al., Cancer Res. 67(1):262-8, 2007); fractionated low-dose radiation (Pogribny et al., Mol Cancer Res. 3(10):553-61, 2005); recombinant human IL -2 (rhIL-2) (Lee et al., Regul Toxicol Pharmacol. 64(2):253-62, 2012); CP-31398 (N′ -[2-[2-(4-methoxyphenyl)ethenyl]-4-quinazolinyl]-N,N-dimethyl-1,3-propanediamine dihydrochloride), a styrylquinazoline that stabilizes the DNA binding conformation of p53 (Johnson et al., Toxicology. 289(2-3):141-50, 2011); synthetic retinoic acid analog, 9-cis-UAB30 [(2E,4E,6Z,8 E)-8-(3′,4′-dihydro-1′(2′H)-naphthalen-1′-ylidene)-3,7-dimethyl-2,4,6-octatrienoic acid], which is used to treat breast cancer (Kapetanovic, Int J Toxicol. 29(2):157-64, 2010) flavopiridol, cyclin-dependent kinase inhibitor, in treating non-small lung cancer (Zveleil, IDrugs. 1(2):241-6, 1998); E-4IB (ethyl-4-isothiocyanatobutanoate) (Tulinska et al., Toxicology 145(2-3):217-25, 2000); 5-fluorouracil (5-FU) and its prodrug 5′-deoxy-5-fluorouridine (5′-DFUR) (Ishikawa et al., Jpn J Cancer Res. 80(6):583-91, 1989); and cyclosporine A (Bennett, J Natl Cancer Inst. 75(5):925-36, 1985), among others.

In some embodiments, the subject has a chronic illness, e.g., a chronic viral illness such as infection with Human Immunodeficiency Virus (HIV), acquired immune deficiency syndrome (AIDS), and AIDS-related complexes; chronic hepatitis (e.g., infection with Hepatitis C or Hepatitis B virus); subacute sclerosing panencephalitis (chronic measles encephalitis); chronic papovavirus encephalitis (progressive multifocal leukoencephalopathy); and Epstein-Barr virus infection.

In some embodiments, the subject has or is at risk of developing an autoimmune disease associated with or as a result of having a reduced numbers of T cells, or of an aberrant T cell repertoire; see, e.g., Datta, S., and Sarvetnick, N. (2009). Lymphocyte proliferation in immune-mediated diseases. Trends Immunol 30, 430-438; Gagnerault, M.-C., Lanvin, O., Pasquier, V., Garcia, C., Damotte, D., Lucas, B., and Lepault, F. (2009). Autoimmunity during thymectomy-induced lymphopenia: role of thymus ablation and initial effector T cell activation timing in nonobese diabetic mice. The Journal of Immunology 183, 4913-4920; Kaminitz, A., Mizrahi, K., Yaniv, I., Stein, J., and Askenasy, N. (2010). Immunosuppressive therapy exacerbates autoimmunity in NOD mice and diminishes the protective activity of regulatory T cells. J Autoimmun 35, 145-152 ; King, C., Ilic, A., Koelsch, K., and Sarvetnick, N. (2004). Homeostatic expansion of T cells during immune insufficiency generates autoimmunity. Cell 117, 265-277; and Zou, L., Mendez, F., Martin-Orozco, N., and Peterson, E. J. (2008). Defective positive selection results in T cell lymphopenia and increased autoimmune diabetes in ADAP-deficient BDC2.5-C57BL/6 mice. Eur J Immunol 138, 986-994.

In some embodiments, the subject has experienced trauma to the thymic region or has had a surgical procedure that impacted the size of the thymus, e.g., cardiothoracic surgery (e.g., in neonates; see, e.g., Eysteinsdottir et al., The influence of partial or total thymectomy during open heart surgery in infants on the immune function later in life. Clin Exp Immunol. May 2004; 136(2):349-355). In some embodiments, the subject has undergone a thymectomy, e.g., to treat cancer, e.g., thymoma, or to treat myasthenia gravis (Manlula et al., Video-assisted thoracic surgery thymectomy for nonthymomatous myasthenia gravis. Chest 2005; 128:3454-3460).

Generally, the methods include administering a therapeutically effective amount of GDF11 as described herein, to a subject who is in need of, or who has been determined to be in need of, such treatment.

As used in this context, to “treat” means to ameliorate at least one symptom of the disorder associated with thymic insufficiency. Often, thymic insufficiency results in a reduction in T cells; thus, a treatment can result in an increase in Tcells. In subjects who have thymic insufficiency in the context of a chronic infection, e.g., viral or bacterial infection, over time, a majority of the T cells will recognize the infectious agent causing the illness (e.g., the virus, bacterium, or other pathogen), and a treatment can result in an increase in the variety of epitopes recognized by the subject's T cells (i.e., a more diverse T cell repertoire). Administration of a therapeutically effective amount of a GDF11 composition described herein for the treatment of a condition associated with thymic insufficiency will result in increased thymic mass and increased levels of nave, newly developed some embodiments, administration of a therapeutically effective amount of a GDF11 composition described herein will result in an increase in numbers of T cells, e.g., levels of CD4+ T and/or levels of naive (CD45RA+CD62L+) T cells, that are persistently (e.g., over a period of weeks to months) above a threshold (e.g., preferably normal) level, e.g., above about 50, 100, 200, 300, 400, or 500 cells/mm³ of whole blood, e.g., more than 50 naive T cells/mm³ or have naive T cells that comprise more than 5% of total T cells by flow cytometry. Thus the methods can include monitoring numbers of T cells, e.g., levels of CD4+ T cells, and/or levels of naive (CD45RA+CD62L+) T cells, or monitoring the numbers of recent thymic emigrating T cells via PCR-based measurement of T cell receptor rearrangement excision circles (Geenen et al., (2003). J. Endocrinol. 176, 305-311) and adjusting or continuing dosing until a threshold level is reached.

GDF11 Compositions-Polypeptides

Growth/differentiation factor 11 (GDF11), also known as Bone morphogenetic protein 11 (BMP-11), is a member of the bone morphogenetic protein (BMP) family and the TGF-beta superfamily. The sequence of the human GDF11 cDNA is available in Genbank at Acc. No. NM _005811.3. The sequence of human GDF11 is as follows:

```
(SEQ ID NO: 1)
    1 mvlaaplllg flllalelrp rgeaaegpaa aaaaaaaaaa agvggerssr papsvapepd
  61 gcpvcvwrqh srelrlesik sqilsklrlk eapnisrevv kqllpkappl qqildlhdfq
121 gdalqpedfl eedeyhatte tvismaqetd pavqtdgspl cchfhfspkv mftkvlkaql
181 wvylrpvprp atvylqilrl kpltgegtag gggggrrhir irslkielhs rsghwqsidf
241 kqvlhswfrq pqsnwgiein afdpsgtdla vtslgpgaeg lhpfmelrvl entkrsrrnl
301 gldcdehsse srccrypltv dfeafgwdwi iapkrykany csgqceymfm qkyphthlvq
361 qanprgsagp cctptkmspi nmlyfndkqq iiygkipgmv vdrcgcs
```

In some embodiments, the methods include administering a polypeptide comprising SEQ ID NO:1, or comprising amino acids 25-407 of SEQ ID NO:1 (as 1-24 likely comprise a signal sequence), or comprising amino acids 299-407 of SEQ ID NO:1 (as the KRSRR sequence immediately preceding position 299 represents a processing protease cleavage site that is common among BMP family members and is likely important for generating the active, processed polypeptide). In some embodiments, the methods include administering a GDF11 polypeptide that is at least 80% identical to the full length of SEQ ID NO:2, or to amino acids 25-407 or 299-407 of SEQ ID NO:1, e.g., at least 85%, 90%, 95%, or 99% identical to SEQ ID NO:1 or to amino acids 25-407 or 299-407 of SEQ ID NO:1. To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). For comparison purposes, a sequence of at least 80% (in some embodiments, about 85%, 90%, 95%, or 100%) of the length of the reference sequence (e.g., SEQ ID NO:1, or amino acids 25-407 or 299-407 of SEQ ID NO:1) is aligned. The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package, using a Blossum 62 scoring matrix with a gap penalty of 12, a gap extend penalty of 4, and a frameshift gap penalty of 5.

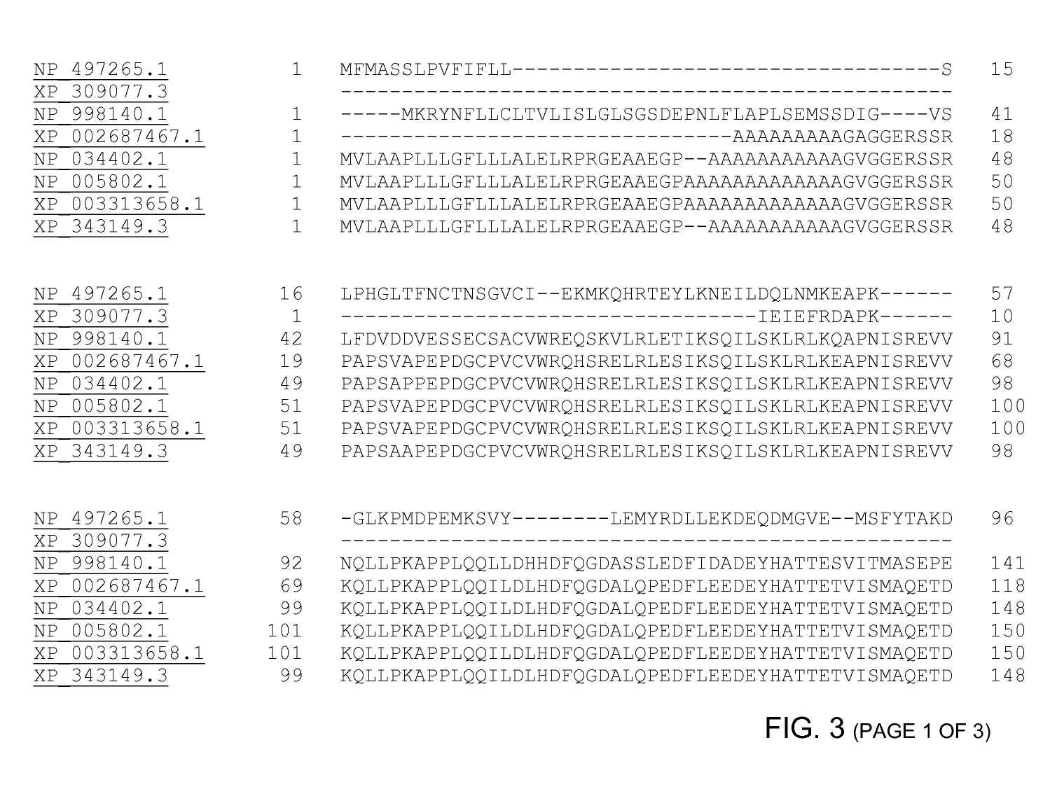

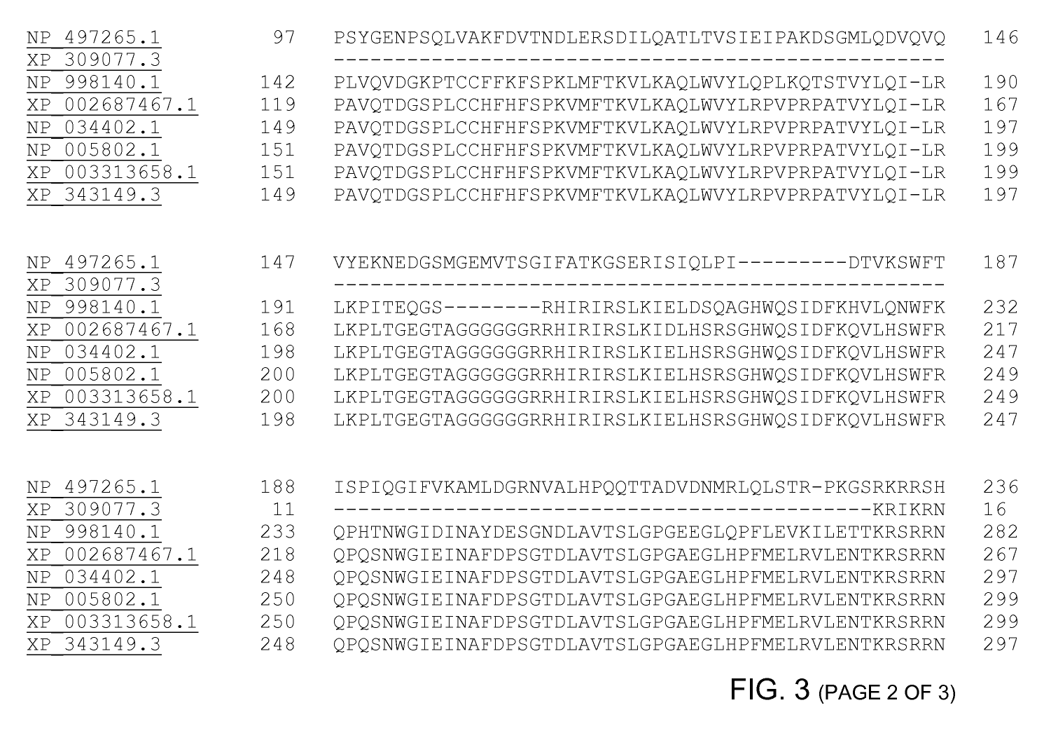

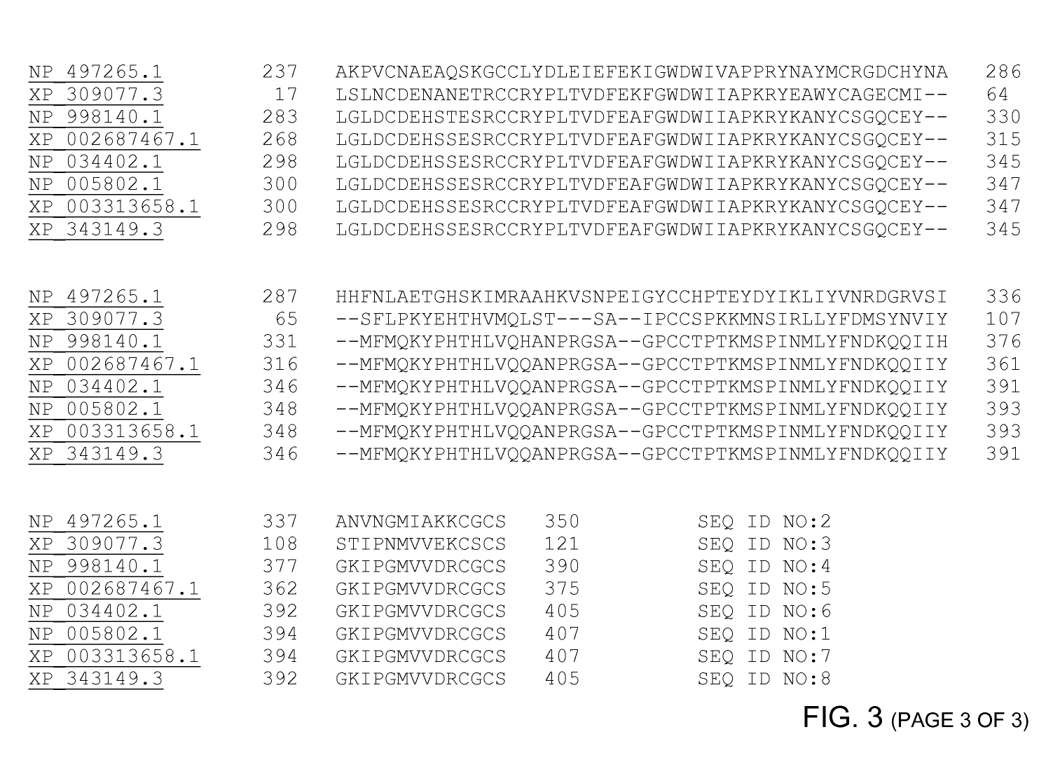

In some embodiments, any variations from SEQ ID NO:1 are outside the region of amino acids 60-287 (a TGFb propeptide region), 299-407, or 313-407 (a TGFb domain) of SEQ ID NO:1. In some embodiments, variations are present in non-conserved regions, which can be identified based on the alignment shown in FIG. 3.

As one of skill in the art will appreciate, it is generally desirable to administer a polypeptide from the same species as the subject to be treated; for example, for a human one would select human GDF11. Other species can also be treated using their own species' GDF11, e.g., as shown in the following Table 1:

TABLE 1

| Protein Acc. | Gene | Organism |
|---|---|---|
| NP_497265.1 | daf-7 | C. elegans |
| XP_309077.3 | AgaP_AGAP005289 | A. gambiae |
| NP_998140.1 | gdf11 | D. rerio |
| XP_002687467.1 | GDF11 | B. taurus |
| NP_034402.1 | Gdf11 | M. musculus |
| NP_005802.1 | GDF11 | H. sapiens |
| XP_003313658.1 | GDF11 | P. troglodytes |
| XP_343149.3 | Gdf11 | R. norvegicus |

An alignment of these sequences is shown in FIG. 3.

GDF11 Compositions-Nucleic Acids

The methods described herein can also include administration of nucleic acids expressing GDF11, e.g., targeted expression vectors for in vivo transfection and expression of GDF11 as described herein, in particular cell types, especially thymic cells, e.g., transplanted thymic epithelial cells, or in thymic horning T cell progenitors, or in other cells that home efficiently to the thymus after transplantation. Expression constructs of such components can be administered in any effective carrier, e.g., any formulation or composition capable of effectively delivering the component gene to cells in vivo. Approaches include insertion of the gene in viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (e.g., lipofectamine) or derivatized (e.g., antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manual. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm, Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral cycle. See, for example, Berkner et al., BiaTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol.158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993).

In addition to viral transfer methods, such as those illustrated above, non-viral methods can also be employed to cause expression of GDF11 in the tissue of a subject. Typically non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol. 116(1):131-135 (2001); Cohen et al., Gene Ther. 7(22):1896-905 (2000); or Tam et al., Gene Ther. 7(21):1867-74 (2000).

In some embodiments, a gene encoding GDF11 is entrapped in Liposomes bearing positive charges on their surface (e.g., lipofectins), which can be tagged with antibodies against cell surface antigens of the target tissue (Mizuno et al., No Shinkei Geka 20:547-551 (1992); PCT publication WO91/06309; Japanese patent application 1047381; and European patent publication EP-A-43075).

In clinical settings, the gene delivery systems for the therapeutic gene can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection (e.g., Chen et al., PNAS USA 91: 3054-3057 (1994)).

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

GDF11 Compositions—Nucleic Acids and Cell Therapy

Levels of GDF11 can also be increased in a subject by introducing into a cell expressing GDF11, e.g., an autologous cell engineered to express exogenous GDF11, e.g., a thymic epithelial cell, or other thymic stromal cell, or a stromal cell derived from another tissue such as skin, or a hematopoietic thymic homing cell such as a common lymphoid progenitor cell or a multipotent progenitor cell (see, e.g., Boehm and Bleul, Trends in Immunology 27(10):477-484 (2006); Dunon and Imhof, Blood, 81(1):1-8 (1993); Zlotoff and Bhandoola, Annals of the New York Academy of Sciences, 1217 (Year in Immunology):122-138 (2011)), comprising a nucleic acid that encodes a GDF11 polypeptide as described herein. The nucleic acid can include any or all of: a promoter sequence, e.g., a promoter sequence from GDF11 or (preferably) from another gene, that drives expression in the cell; an enhancer sequence, e.g., 5′ untranslated region (UTR), e.g., a 5′ UTR from GDF11 or from another gene, a 3′ UTR, e.g., a 3′ UTR from a GDF11 gene or from another gene; a signal sequence; a polyadenylation site; or an insulator sequence. The cell can then be introduced into the subject.

Primary and secondary cells to be genetically engineered can be obtained from a variety of tissues and can include cell types that can be maintained and propagated in culture. For example, primary and secondary cells useful in the present methods include fibroblasts, keratinocytes, epithelial cells (e.g., mammary epithelial intestinal epithelial cells), endothelial cells, muscle cells (myoblasts) and precursors of these somatic cell types. Primary cells are preferably obtained from the individual to whom the genetically engineered primary or secondary cells will be administered (i.e., are autologous). However, primary cells can also be obtained from a donor (i.e., an individual other than the recipient).

The term “primary cell” includes cells present in a suspension of cells isolated from a vertebrate tissue source (prior to their being plated, i.e., attached to a tissue culture substrate such as a dish or flask), cells present in an explant derived from tissue, both of the previous types of cells plated for the first time, and cell suspensions derived from these plated cells. The term “secondary cell” or “cell strain” refers to cells at all subsequent steps in culturing. Secondary cells are cell strains which consist of primary cells which have been passaged one or more times.

Primary or secondary cells of vertebrate, particularly mammalian, origin can transfected with the exogenous GDF11 nucleic acid sequence, and produce the encoded product stably and reproducibly in vitro and in vivo, over extended periods of time.

A heterologous nucleic acid can also be a regulatory sequence, e.g., a promoter, which causes expression, e.g., inducible expression or upregulation, of an endogenous sequence. An exogenous nucleic acid sequence can be introduced into a primary or a secondary cell by homologous recombination as described, for example, in U.S. Pat. No. 5,641,670, the contents of which are incorporated herein by reference. The transfected primary or secondary cells can also include DNA encoding a selectable marker, which confers a selectable phenotype upon them, facilitating their identification and isolation.

Vertebrate tissue can be obtained by standard methods such a punch biopsy or other surgical methods of obtaining a tissue source of the primary cell type of interest. For example, a biopsy can be used to obtain skin, as a source of cells, e.g. fibroblasts. A mixture of primary cells can be obtained from the tissue, using known methods, such as enzymatic digestion or explanting. If enzymatic digestion is used, enzymes such as collagenase, hyaluronidase, dispase, pronase, trypsin, elastase and chymotrypsin can be used.

The resulting primary cell mixture can be transfected directly, or it can be cultured first, removed from the culture plate and resuspended before transfection is carried out. Primary cells or secondary cells are combined with exogenous nucleic acid sequence to, e.g., stably integrate into their genomes, and treated in order to accomplish transfection. As used herein, the term “transfection” includes a variety of techniques for introducing an exogenous nucleic acid into a cell including calcium phosphate or calcium chloride precipitation, microinjection, DEAE-dextrin-mediated transfection, lipofection or electroporation, all of which are routine in the art.

Transfected primary or secondary cells undergo sufficient numbers of doubling to produce either a clonal cell strain or a heterogeneous cell strain of sufficient size to provide the therapeutic protein to an individual in effective amounts. The number of required cells in a transfected clonal heterogeneous cell strain is variable and depends on a variety of factors, including but not limited to, the use of the transfected cells, the functional level of the exogenous DNA in the transfected cells, the site of implantation of the hansfected cells (for example, the number of cells that can be used is limited by the anatomical site of implantation), and the age, surface area, and clinical condition of the patient.

The transfected cells, e.g., cells produced as described herein, can be introduced into an individual to whom the product is to be delivered. Various routes of administration and various sites (e.g., renal sub capsular, subcutaneous, central nervous system (including intrathecal), intravascular, intrahepatic, intrasplanchnic, intraperitoneal (including intraomental), intramuscularly implantation) can be used. Once implanted in an individual, the transfected cells produce the product encoded by the heterologous DNA or are affected by the heterologous DNA itself. For example, an individual who suffers from thymic insufficiency is a candidate for implantation of cells producing GDF11.

Combination Treatments

In some embodiments, the methods described herein are employed in combination with one or more other treatment modalities, e.g., treatment modalities for the regeneration of the thymus or parts thereof, e.g., as described in Lynch, H. E., Goldberg, G. L., Chidgey, A., van den Brink, M. R. M., Boyd, R., and Sempowski, G. D. (2009). Thymic involution and immune reconstitution. Trends Immunol 30, 366-373. Exemplary methods include castration (Griffith et al., (2011) Aging Cell 11, 169-177); administration of keratinocyte growth factor (KGF; Min et al., (2007). Blood 109, 2529-2537); administration of ghrelin (Dixit et al., (2007). J Clin Invest 117, 2778-2790); administration of human growth hormone (Goya et al., (1992). Brain Behav. Immun. 6, 341-354); and administration of interleukin-22 (Dudakov et al., (2012). Science 336, 91-95). Thus the methods can include administering GDF11 in combination with KGF, ghretin, human growth hormone, and/or IL-22, e.g., administered simultaneously, e.g., in the same or different pharmaceutical composition and at substantially the same time (e.g., within 30-60 minutes of each other), or administered sequentially, e.g., in one or more doses.

In some embodiments, the methods also include transplanting thymic tissues into the subject, e.g., where the subject tacks a thymus altogether, e.g., due to genetic reasons, e.g., DiGeorge syndrome, or as a result of other causes including those listed above. In some embodiments, allogeneic thymic tissue is transplanted, e.g., as described in Markert et al., Clin Immunol. May 2010; 135(2):236-46; Markert et al., N Engl J Med. 1999 Oct. 14; 341(16):1180-9; Markert et al., Blood. 2004 Oct. 15; 104(8):2574-81; Markert et al., Blood. 2007 May 15; 109(10):4539-47; and Chinn and Markert, J Allergy Clin Immunol. June 2011; 127(6):1351-5. In some embodiments, the transplant includes a thymic epithelial cell, or other thymic stromal cell, or a stromal cell derived from another tissue such as skin, or a hematopoietic thymic homing cell such as a common lymphoid progenitor cell or a multipotent progenitor cell (see, e.g., Boehm and Bleul, Trends in Immunology 27(10):477-484 (2006); Dunon and Imhof, Blood, 81(1):1-8 (1993); Zlotoff and Bhandoola, Annals of the New York Academy of Sciences, 1217 (Year in Immunology):122-138 (2011)). In some embodiments immune suppressive treatments are also administered, as described in the above references.

T cell presence in tumors is typically associated with immune surveillance and improved patient survival (Zhang et al. 2003. The New England Journal of Medicine 348:203-213; Fridman et al., 2011. Cancer research 71:5601-5605; Pages et al. 2005. The New England journal of medicine 353:2654-2666; Yu et al., 2009. Nature reviews. Cancer 9:798-809; Yu et al., 2007. Nature Reviews. Immunology 7:41-51; Schreiber et al., 2011, Science 331:1565.-1570; Vesely et al., 2011. Annual Review of Immunology 29:235-271). Consequently, immunotherapy using blockade of negative regulators of T cells function is an especially attractive approach (Hodi et al., The New England journal of medicine 2010 363:711-723; Topalian et al., 2012. The New England Journal of Medicine 366:2443-2454). Unlike genetic lesion-specific therapies, immunotherapy has potential for targeting tumors irrespective of driver oncogene mutation status. Thus in some embodiments, the methods also include administering an immunotherapy to the subject, e.g., one or more therapies that promote anti-cancer immunity, including administering one or more of: dendritic cells or peptides with adjuvant, immune checkpoint inhibitors, DNA-based vaccines, cytokines (e.g., IL-2), cyclophosphamide, agonists of OX40 (OX40; CD134), anti-interteukin-2R immunotoxins, and/or antibodies such as anti-CD137, anti-PD1, or anti-CTLA-4; see, e.g., Krüger et al., Histol Histopathol. June 2007; 22(6):687-96; Eggermont et al., Semin Oncol. October 2010; 37(5):455-9; Klinke D J 2nd, Mol Cancer. 2010 Sep. 15; 9:242; Alexandrescu et al., J Immunother. July-August 2010; 33(6):570-90; Moschella et al., Ann N.Y. Acad Sci. April 2010; 1194:169-78; Ganesan and Bakhshi, Natl. Med J India. January-February 2010; 23(1):21-7; Golovina and Vonderheide, Cancer J. July-August 2010; 16(4):342-7; Hodi et al., The New England journal of medicine 2010 363:711-723; Pentcheva-Hoang et al., Immunological Reviews 2009 229:67-87; Brahmer et al., Journal of Clinical Oncology 2010 28:3167-3175; Lynch et al., Journal of Clinical Oncology 2012 30(17):2046; Weber, Current Opinion in Oncology 2011 23:163-169; Weber, Seminars in Oncology 2010 37:430-439; Topalian et al., 2012. The New England Journal of Medicine 366:2443-2454; and Higano et al., Cancer 2009 115:3670-3679. In some embodiments, the methods include administering a composition comprising tumor-pulsed dendritic cells, e.g., as described in WO2009/114547 and references cited therein. Additional examples of immunotherapies include virus-based anti-cancer vaccines (e.g., adenovirus), formulations of Toll-like Receptor or RIG-I-like receptor ligands, Adoptive T cell therapy or other cell types. In some embodiments the immunotherapy is selected from the group consisting of BiovaxID (an autologous vaccine containing tumor-specific idiotype proteins from individual patient's lymphoma cells conjugated to keyhole limpet hemocyanin (KLH)); Provenge sipuleticet-T (an FDA-approved example of the use of autologous dendritic cells); Yervoy (a mAb against CTLA4 (CD152), approved in 2011 for metastatic melanoma); trernelimumab (formerly ticilimumab, an anti-CTLA-4 mAb); IMA901 (a vaccine containing 10 tumor-associated peptides (TUMAPs)), alone or in combination with Sutent (a small molecule VEGF receptor tyrosine kinase inhibitor); GV1001 (a peptide vaccine with the sequence of human telomerase reverse transcriptase (hTERT), from Kael-Gernvax); Lucanix belagenpurnatecel-L (four NSCLC cell lines carrying antisense oligonucleotides against transforming growth factor beta 2 (TGFB2)); Stimuvax (a liposomal vaccine containing a synthetic 25-amino acid peptide sequence from mucin 1 (MUC1; CD227)); Allovectin velimogene aliplasmid (a DNA plasmid encoding major histocompatibility complex (MHC) class I B7 (HLA-B7) complexed with lipid); BMS-936558 (ONO-4538) (a human mAb against PD-1); BMS-936559 (formerly MDX-1105) (a human mAb against PD-L1); Zelboraf (vemurafenib, an oral small molecule inhibitor of the oncogenic BRAF V600E mutation); Votrient (pazopanib, a small molecule VEGF receptor tyrosine kinase inhibitor); ISF35 or Lucatumumab (HCD122) (mAbs against CD40); GVAX (an allogeneic cancer vaccine engineered to secrete granulocyte macrophage-colony stimulating factor (GM-CSF)), See, e.g., Flanagan, “Immune Springboard,” Biocentury, Jun. 18, 2012 A5-A10 (2012), available at biocentury.com. In some embodiments, the immunotherapy comprises administration of an agent that effects CTLA4 blockade (e.g., Ipilumumab BMS), PD1-blockade (e.g., BMS-936558, BMS; CT-011, Curetech; MK-3475, Merck), CD137 activation (e.g., BMS-663513, BMS), PD-L1 blockade (e.g., BMS-936559, BMS), CD40 activation (e,g. CP-870893, Pfizer) and autologous dendritic cells (e.g., Provenge).

Inhibition of WFIKKN2

In some embodiments of the methods described herein, in addition to or as an alternative to administration of GDF 11, the methods include administration of an inhibitor of WFIKKN2 (also known as WAP, follistatin/kazal, immunoglobulin, kunitz and netrin domain containing 2), which binds to and inhibits GDF11, see, e.g., Hill, J. J., Qiu, Y., Hewick, R. M., and Wolfman, N. M. (2003). Regulation of myostatin in vivo by growth and differentiation factor-associated serum protein-1: a novel protein with protease inhibitor and follistatin domains. Mol. Endocrinol. 17, 1144-1154; and Kondás, K., Szláma, G., Nagy, A., Trexler, M., and Patthy, L. (2011). Biological functions of the WAP domain-containing multidomain proteins WFIKKN1 and WFIKKN2. Biochem. Soc. Trans. 39, 1416-1420. Inhibitors of WFIKKN2 are known in the art and include inhibitory nucleic acids targeting WFIKKN 2, e.g., antisense, siRNA, or shRNA. The nucleic acid sequence of human WFIKKN2 is available in genbank at NM_175575.5, the protein sequence is NP_783165.1.

Pharmaceutical Compositions and Methods of Administration

The methods described herein include the manufacture and use of pharmaceutical compositions that include GDF11 as an active ingredient, for use in a method of treatment as described herein.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., keratinocyte growth factor (KGF); ghrelin; human growth hormone; interleukin-22 (IL-22); and sex steroid inhibitors (e.g., androgen activity inhibitors and estrogen activity inhibitors, e.g., LHRH agonists, LHRH antagonists, anti-LHRH receptor antibodies, anti-LHRH vaccines, anti-androgens, anti-estrogens, selective estrogen receptor modulators (SERMs), selective androgen receptor modulators (SARMs), selective progesterone response modulators (SPRMs), ERDs, aromatase inhibitors; see, e.g., US 2008/0279812 and WO1993010741).

Pharmaceutical compositions are typically formulated to be compatible with its intended route of administration. Examples of routes of administration include parenteral, e.g., intravenous, intradermal, subcutaneous, oral or nasal (e.g., inhalation), transdermal or topical, transmucosal, and rectal administration. For the present methods, parenteral administration is preferred.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains abasic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No, 4,522,811.

The pharmaceutical compositions can be included in a container, pack, or dispenser together with instructions for administration.

Dosage

An “effective amount” is an amount sufficient to effect beneficial or desired results. For example, a therapeutic amount is one that achieves the desired therapeutic effect, i.e., results in a desired or sufficient increase in thymic mass, thymic function, or level or variety of T cells. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximai inhibition of symptoms) as determined in cell culture. Such information can be used to more accurately determine useful doses in humans. Levels in plasma may be measured, for example, by high performance liquid chromatography.

### EXAMPLES

The invention is further described in the following examples, which do not limit the scope of the invention described in the claims.

### Example 1

### GDF11 Promotes Thymic Regeneration

Female 24-month old C57/BL6 mice were injected daily with GDF11 (0.1 mg/kg) or saline (vehicle control) by intraperitoneal injection for 30 d. The thymuses of the mice were then mashed to create a single cell suspension, or were frozen in tissue freezing medium and sectioned into 5 um sections. The total cellularity of each thymus was determined using a hemacytometer. These results indicate that daily, intraperitoneal injection of GDF11 into aged mice resulted in dramatically increased thymic cellularity (FIG. 1).

### Example 2

### GDF11 Acts Directly on Thymic Epithelial Cells

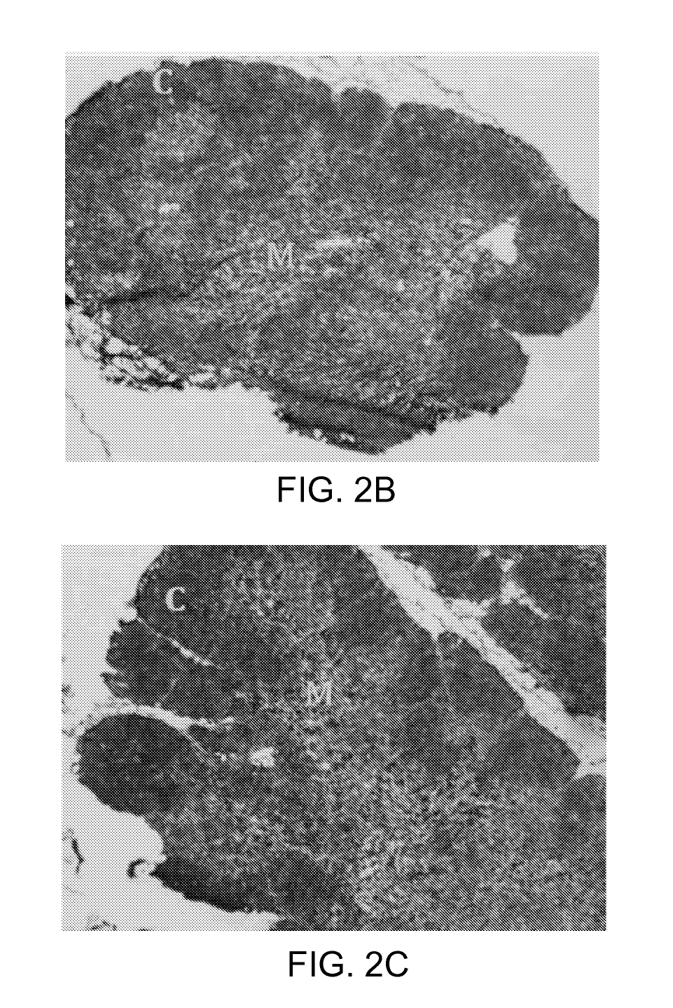

As described in Example 1, daily, intraperitoneal injection of GDF11 into aged mice resulted in dramatically increased thymic cellularity (FIG. 1). One important question regarding the GDF11 effect is: which cells within the thymus does GDF11 act upon? GDF11 could act directly upon the developing T cells, on the thymic stromal cells, or alternatively, GDF11 could act upon the thymus indirectly, for example, by stimulating hormone release from a separate tissue. To distinguish these possibilities, the effects of GDF 11 directly on thymic stromal cells, developing T cells, and on the thymus as a whole, were assessed. Tissue sections of the thymuses from GDF11 treated mice were stained with hernatoxylin and eosin; these showed that the treated thymuses had regenerated both medullary and cortical thymic zones, and had a normal thymic morphology (FIG. 2).

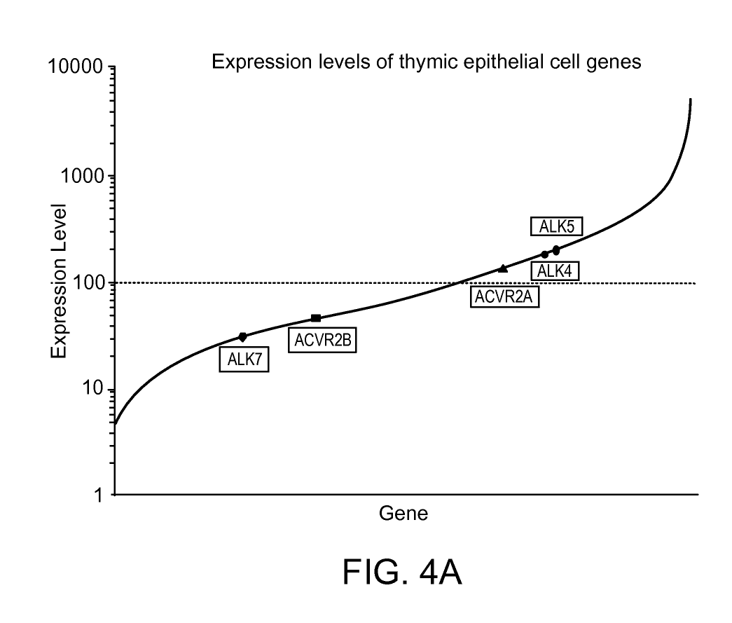

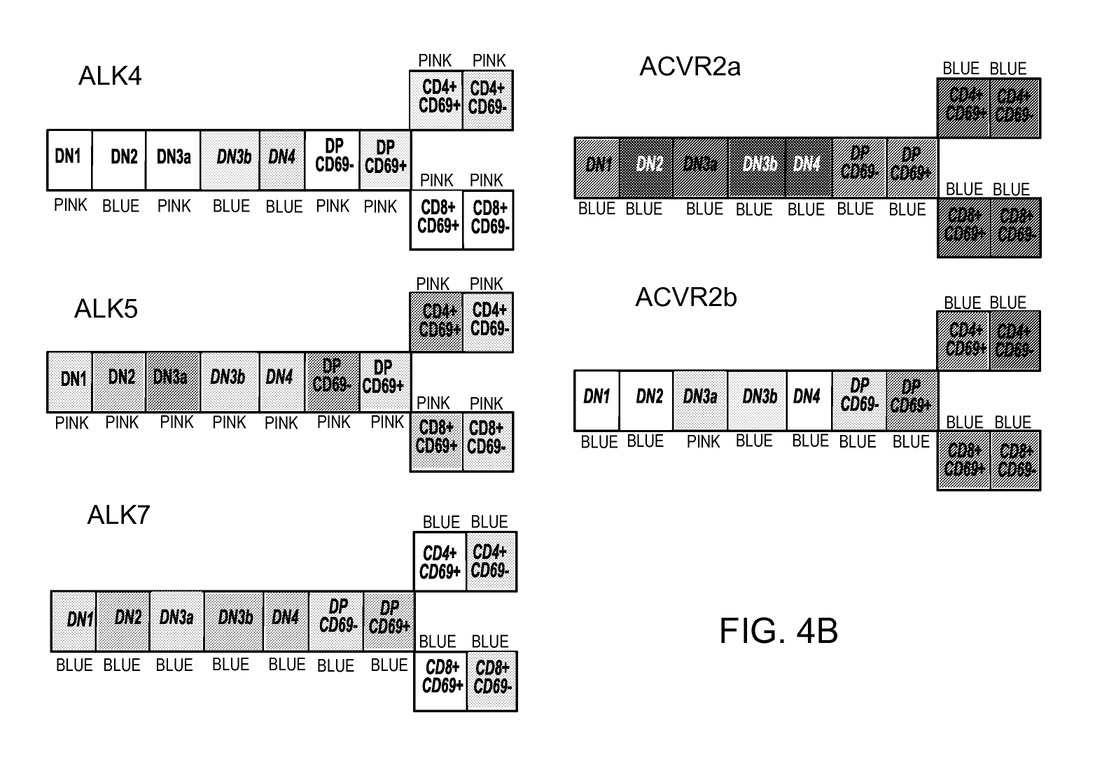

GDF11 has been shown to bind and signal through several overlapping sets of heterodimeric receptors composed of type I receptor subunits Allk4, Alk5, and Alk7, in combination the type 2 receptor subunits Acvr2 and Acvr2b (Oh and Li, Genes & Development 11, 1812-1826 (1997); Andersson et al., EMBO Rep. 7, 831-837 (2006)). Gene expression array analysis was performed using mRNA isolated from purified subpopulations of thymic epithelial cells. These gene expression arrays showed that several of the receptors capable of binding to GDF11 were expressed on thymic epithelial cells (TECs) (FIG. 4A). In this figure, the expression levels of each gene from thymic epithelial cells (as estimated from the annealing of prepared, amplified RNA to the Affymetrix Exon chips) was determined, and the normalized, relative gene expression values were graphed. Each point on the graph shows the average expression level of one gene in thymic epithelial cells. The genes that encode potential GDF11 receptors are shown, With an expression level of “100”, arbitrarily chosen as being expressed at a detectable level, thymic epithelial cells express measurable levels of both type 1 (ACVR2A) and type 2 (ALK4, and ALK5) receptors. This strongly suggests that thymic epithelial cells should be able to bind and respond to GDF11. Similarly, gene expression arrays of T cell progenitors were performed. There are several subpopulations of developing T cell progenitors within the thymus, as represented by boxes (FIG. 4b), The level of expression of GDF11 receptors was assessed in each subpopulation, and displayed as either pink (highly expressed) or blue (not expressed, or low expressed) colored boxes. These gene expression arrays showed that developing T cells change their expression of GDF11 receptors as they develop, but that in general, they can express ALK4 and ALK5, and that early stage T cell progenitors (which are the most proliferative) also express ACVR2b. These results indicate that GDF11, as well as other TGFβ family members, might act upon multiple thymic cell types (FIGS. 4A and 4B).

Age-related changes in TEC may underlie age-related thymic involution; similarly, factors that drive TEC growth can support thymic regeneration (Heng et al., 2010). Therefore, the ability of GDF11 to stimulate TEC growth was assessed. TEC were isolated from thymuses of young mice using standard techniques (Gray et al., (2002), J Immunol Methods 260:15-28) and sorted using a Becton Dickinson FACSAria cell sorter. Thymic epithelial cells were cultured for up to two weeks in the presence or absence of GDF11, The results indicated that GDF11 directly promoted the survival and proliferation of TEC in culture. This finding suggests that direct action of GDF11 on TEC may be responsible for the GDF11-induced thymic growth seen in vivo (FIG. 1).

Since the expression analysis indicates that multiple subsets of developing T cells also express receptors that can bind and respond to GDF11, the direct effects of GDF11 on thymocyte growth in vitro are tested by culturing sorted thymocytes on the thymocyte-supportive OP9-delta in the presence or absence of GDF11. Given the dramatic effects of GDF11, it is possible that it acts on both thymocytes as well as the TEC.

In addition to TEC and developing T cells, there are multiple other cell types within the thymus that might act as important targets for GDF 11, including fibroblasts, endothelial cells, macrophages and dendritic cells. In order to include the potential of all these cell types to mediate GDF11-induced thymic growth, fetal thymic organ cultures (FTOC) are performed in the presence or absence of GDF11 to test whether GDF11 promotes additional FTOC growth. As GDF11 mRNA is undetectable within the thymus and seems to be acting hormonally within the mouse, endogenously expressed GDF11 within the FTOCs is not likely to confound the conclusions.

### ADDITIONAL REFERENCES

Allen et al., (1992). Mol Cell Biol 12, 2758-2768.

Esquela and Lee, (2003). Dev Biol 257, 356-370.

Gordon et al., (2010). Dev Biol 339, 141-154.

Hauri-Hohl et al., (2008). Blood 112, 626-634.

Hill et al., (2003). Mol. Endocrinol, 17, 1144-1154.

McPherron et al., (1999). Nature Genetics 22, 260-264.

Richie Ehrlich et al., (2011). Blood 117, 2618.

Serwold et al., (2007). The Journal of Immunology 179, 928.

Serwold et al., (2009). Blood 113, 807-815.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating thymic insufficiency in a mammalian subject, the method comprising administering to the subject a therapeutically effective amount of a composition comprising a growth differentiation factor 11 (GDF11) polypeptide.

2. A method of treating thymic insufficiency in a mammalian subject, the method comprising administering to the subject a therapeutically effective amount of a composition comprising a nucleic acid encoding a growth differentiation factor 11 (GDF11) polypeptide.

3. A method of treating thymic insufficiency in a mammalian subject, the method comprising administering to the subject a therapeutically effective amount of a composition comprising a cell expressing an exogenous nucleic acid encoding a growth differentiation factor 11 (GDF 11) polypeptide.

4. Use of a GDF 11 polypeptide or nucleic acid encoding a GDF11 polypeptide for treating thymic insufficiency in a subject, or for the manufacture of a medicament for the treatment of thymic insufficiency in a subject.

5. The method or use of any of the preceding claims, wherein the subject is a human.

6. The method or use of claim 4, wherein the polypeptide comprises an amino acid sequence that is at least 80%, 90%, or 95% identical to the full length of SEQ ID NO:1, or at least 80%, 90%, or 95% identical to amino acids 25-407 or 299-407 of SEQ ID NO:1.

7. The method of use of any claims 1-4, wherein the subject has lymphopenia.

8. The method or use of claim 7 wherein the subject has levels of CD4+ T cells that are persistently below a threshold level.

9. The method or use of claim 8, wherein the subject has levels of CD4+ T cells that are below about 50, 100, 200, 300, 400, or 500 cells per mm³ of whole blood.

10. The method or use of any claims 1-4, wherein the subject has cancer.

11. The method or use of claim 10, wherein the subject has been treated with a thymotoxic chemotherapy.

12. The method or use of claim 10, further comprising administering an immunotherapy to the subject.

13. The method or use of any claims 1-4, wherein the subject has a chronic illness.

14. The method or use of claim 13, wherein the chronic illness is a chronic viral illness such as infection with Human Immunodeficiency Virus (HIV), acquired immune deficiency syndrome (AIDS), and AIDS-related complexes; chronic hepatitis (e.g., infection with Hepatitis C or Hepatitis B virus); subacute sclerosing panencephalitis (chronic measles encephalitis); chronic papovavirus encephalitis (progressive multifocal leukoencephalopathy); or Epstein-Barr virus infection.

15. The method or use of any claims 1-4, wherein the subject has an autoimmune disease associated with reduced numbers of T cells.

16. The method or use of any claims 1-4, wherein the subject has experienced trauma to the thymic region or has had a surgical procedure that decreased the size of the thymus.

17. The method or use of claim 16, wherein the subject has had cardiothoracic surgery.

18. The method or use of claim 17, wherein the subject is a neonate.

19. The method or use of any claims 1-4, wherein the subject is over the age of 50.

20. The method or use of claim 19, wherein the subject at least 60, 70, 80, or 90 years of age.

21. The method or use of any of claims 1-4, wherein the GDF11 is administered in combination with a treatment comprising castration; administration of keratinocyte growth factor; administration of ghrelin; administration of human growth hormone; and administration of interleukin-22.
